(19) United States
(12) Patent Application Publication
(10) Pub. No.: US 2009/0214440 A1
(43) Pub. Date: Aug. 27, 2009

(54) Method and device for examining a biological tissue

(76) Inventors: Christoph Böhme, Salt Lake City, UT (US); Martin Hoheisel, Erlangen (DE); Klaus Lips, Berlin (DE); Marcus Pfister, Bubenreuth (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/918,807

(22) PCT Filed: Apr. 13, 2006
(86) PCT No.: PCT/EP2006/061577

§ 371 Date: Mar. 3, 2009

(30) Foreign Application Priority Data
Apr. 18, 2005 (DE) 10 2005 017 817.0

(51) Int. Cl. A61B 5/055; G01R 33/32

(52) U.S. Cl. 424/9.37; 324/304

(57) ABSTRACT

The invention relates to a method and a device for analyzing a biological tissue, whereby a luminescence light of a luminescence substance is detected. The aim of the invention is to increase the precision and reliability of the analysis. To this end, a permutation symmetry imbalance is generated in the tissue by a magnetic field, the permutation symmetry imbalance is modified at a pre-determined location by a magnetic alternating field, and the luminescence light is detected according to the pre-determined location.

### CROSS REFERENCE TO RELATED APPLICATIONS

This application is the US National Stage of International Application No. PCT/EP2006/061577 filed Apr. 13, 2006 and claims the benefits thereof. The International Application claims the benefits of German application No. 10 2005 017 817.0 filed Apr. 18, 2005, both of the applications are incorporated by reference herein in their entirety.

### FIELD OF THE INVENTION

The invention relates to a method and a device for examining a biological tissue.

### BACKGROUND OF THE INVENTION

Umar Mahmood et al., “Near Infrared Optical Imaging of Protease Activity for Tumor Detection”, Radiology 1999, 213: 866-870, discloses a method and a device for detecting tumors in mice. To detect the tumor, light is detected by a fluorogen that is activated by tumor proteins. A disadvantage thereof is that tumors or lesions located deep in the tissue cannot be detected safely and reliably due to the blurring caused by the scattering of the light in the tissue.

A. Wall et al., “Designing a multi-channel device for optical fluorescence imaging”, RöFo 2003, VO46.8, discloses a multi-channel device for optical fluorescence imaging. In this system, fluorochromes or fluorescent proteins in a tissue are excited with light from the near infra-red (NIR) range, red, blue and green light, until they fluoresce. The fluorescent light is filtered with emission filters and detected with a CCD camera. As a result of the scattering of the fluorescent light in the tissue, it is not possible to detect fluorescent light from deep layers of tissue with sufficient precision. Tumors or lesions located deep in the tissue cannot be detected safely and reliably.

Vasilis Ntziachristos et al., “Differential diffuse optical tomography”, Optics Express 08.11.1999, Vol. 5, No. 10, pages 230-242, discloses a tomographic method in which images of tissue are generated using differences in the absorption of light in the tissue, caused by a contrast agent. A disadvantage of the method is the limited local resolution caused by the extensive light scattering in the tissue. As a result thereof, the achievable local resolution of the tomographic images is limited.

Furthermore, X. Wang et al., “Noninvasive laser-induced photoacoustic tomography for structural and functional in vivo imaging of the brain”, Nature Biotechnology, Vol. 21, Number 7, July 2003, pages 803 to 806 discloses a photoacoustic tomography method. In this method, a tissue, such as a part of a rat's brain, is optically excited. Based on the photo-acoustic effect, the method generates different acoustic waves according to the nature of the tissue. An image of the tissue can be reconstructed using these waves. The acoustic connection between a receiver and the tissue that is required to carry out the photoacoustic tomographic method is expensive.

Conventional spin resonance devices allow the inner structure of a tissue to be examined and images of the tissue to be reconstructed. It is possible, however, that where there is a poor contrast, small lesions, tumors or suchlike cannot be detected precisely and reliably.

### SUMMARY OF THE INVENTION

The object of the invention is to overcome the disadvantages of the prior art. In particular, the aim is to provide a method and a device with which the inner structure of a biological tissue can be examined precisely and reliably. Furthermore, the aim is to provide a method and a device for analyzing a biological tissue with which precise diagnoses can be made in particular.

This object is achieved by the features of the independent claims. Useful embodiments of the invention are derived from the dependent claims.

According to the invention, a method is provided comprising the following steps:

a) Generation of a magnetic field in the tissue so that a permutation symmetry imbalance is generated in a luminescent substance located in the tissue,
b) Irradiation of the tissue using continuous or pulsed electromagnetic radiation suitable for stimulating a luminescence of the luminescent substance,
c) Generation of a continuous or pulsed magnetic alternating field suitable for modifying the permutation symmetry imbalance at a given location in the tissue and
d) Detection of a luminescent light generated by the luminescence, depending on the location of the modification in the permutation symmetry imbalance.

As described by Böhme and Lips in “Theory of the time-domain measurement of spin-dependent recombination with pulsed electrically detected resonance” in Physical Review B68, 245105, 2003, pages 1 to 19, there is an imbalance between groups of spin pairs with different permutation symmetries when there is an asymmetry in the make-up of coupled spin pairs, such as, for example, nuclear spin/electron spin or electron spin/electron spin pairs. This imbalance is referred to in the above by the term “permutation symmetry imbalance”.

By modifying the permutation symmetry imbalance at a given location it is possible to locally influence the luminescence of the luminescent substance. The influencing of the luminescence results in a modification in the intensity of the luminescent light detected. The influencing of the luminescence can be detected with a high degree of precision. The luminescent light detected can be used as a yardstick for the influencing of the luminescence. From the luminescent light it is possible to make statements on and/or draw conclusions regarding the inner structure of the tissue. The inner structure of the tissue can be examined precisely and reliably. For example, lesions and defects in the tissue can be located with particular precision. Safe diagnoses can be made using the analytical results obtained using the method.

In the context of the present invention, the term “tissue” is also understood to mean a part or organ of a body, especially mammalian, in particular human. In the examination a section or the whole tissue, in particular a surface layer or the inner part of the tissue can be examined.

The term “luminescence” is understood to mean in particular phosphorescence, fluorescence and all atomic or molecular radiation processes generated by electron transfers.

According to one embodiment of the invention, the luminescent light is detected after step b) and before step c) and in step d) a modification of the luminescent light caused by the magnetic alternating field is detected depending on the location of the modification in the permutation symmetry imbalance. Testing errors can be avoided or corrected since the luminescent light is detected before and after the modification to the permutation symmetry imbalance. For example, errors caused by a reduction in the overall intensity of the luminescent light can be avoided or corrected. Such errors can be caused, for example, by a reduction in the concentration or quantity of the luminescent substance or by its becoming degraded. The intensity of the luminescent light detected in each case before the modification to the permutation symmetry imbalance can be used as a benchmark for standardizing the intensities. Thus the precision of the examination can be increased.

According to a further embodiment of the invention, at least one monochromatic radiation source is used to generate the electromagnetic radiation. By using a monochromatic radiation with a given energy level, a luminescence excitable by this energy can be excited in a targeted manner. It is also possible to use a plurality of monochromatic radiation sources. For example, the luminescent substance can comprise a plurality of excitation energies or two or a plurality of luminescent substances with different excitation energies can be used. With different excitation energies, energy-dependent differences in the influencing of luminescence by the alternating field or in the scattering properties of the tissue can be detected. Additional information about the tissue and the inner structure thereof can be obtained from the differences.

The luminescence is preferably a fluorescence and the fluorescent substance located in the tissue is selected from the following group: fluorophores, fluorochromes, fluorogens, fluorescent molecules, fluorescent proteins, such as the known green fluorescent proteins which are also referred to as GFPs. In the case of luminescent substances it is known that said substances accumulate locally to different extents depending on the inner structure of the tissue and/or bind to specific locations in the tissue and/or can be activated only in certain tissue regions. By selecting an appropriate luminescent substance, the precision and local resolution of the method can be improved.

According to a particularly advantageous embodiment, light having a wavelength of between 200 nm and 2000 nm, preferably between 650 nm and 800 nm, is used for electromagnetic radiation. With light in these wavelength regions it is possible to avoid the biological tissue being damaged by the irradiation. Furthermore, such light has a particularly great depth of penetration for biological tissue. A particularly great depth of penetration makes it possible to examine with particular precision the inner structure of the tissue, in particular layers of tissue located at a deep level.

According to one embodiment of the invention, the detection in step d) is preceded by filtering of the luminescent light. It is possible to use filters which are essentially penetrated only by luminescent light. Backscattered light or light not generated by luminescence can be suppressed. As a result of the suppression thereof, the luminescent light can be detected with greater precision.

According to a further embodiment of the invention, a CCD camera, an optical sensor for integral detection of the luminescent light, a photodiode, a photoresistor, a phototransistor, a photomultiplier, and a pyroelectric detector are used. It is also possible to use a different technical device to detect the luminescent light. Additional local information can also be obtained from an intensity distribution of the luminescent light generated with the CCD camera. The local resolution of the method can be improved using the local information. The luminescent light can also be detected in an integral manner, however. A greater intensity is available. Integral detection is particularly advantageous in cases where the luminescent light has a lower intensity and an intensity distribution cannot be determined with sufficient precision.

According to one embodiment of the invention, a gradient field is used as a magnetic field. A modification of the permutation symmetry imbalance occurs when an alternating field that fulfils the known condition of resonance is irradiated. The condition of resonance is fulfilled, for example, if the frequency of the alternating field corresponds to the Larmor frequency of spins in the magnetic field. In a gradient field, the condition of resonance is a function of the location in the tissue. As a result of the size of the gradient field and the gradient intensity being known, an alternating field that fulfils the condition of resonance at a given location can be radiated into the tissue. The detection of the luminescent light or modification thereof can be simplified depending on the location.

To generate the magnetic field or the gradient field and the alternating field, it is possible to use magnetic coils and/or permanent magnets. The alternating field is preferably generated by means of magnetic coils. The alternating field can be radiated into the tissue in such a way that it acts upon the entire tissue. It is also possible, however, to radiate the alternating field only onto a given limited area located in the tissue. The position of the area can also be used as additional local information to improve the local resolution of the method.

A further embodiment of the invention makes provision for generation, modification, irradiation and/or detection means inserted into a cavity located in the tissue or leading to the tissue can be used to generate and/or modify the permutation symmetry imbalance and/or to irradiate the tissue and/or detect luminescence. This allows an examination of the tissue using a minimally invasive method, for example, via a vein, the trachea or the intestine. The generation, modification, irradiation and/or detection means can be inserted into the cavity by using a catheter, a probe or such like, for example. Using incoming lines, outgoing lines, control lines and suchlike connected to the generation, modification, irradiation and/or detection means, the function and/or movement thereof in the cavity can be controlled manually or even automatically. The generation, modification, irradiation and/or detection means can also be provided as units that are separate from one another or combined in any desired combinations. The generation, modification, irradiation and/or detection means can be combined in particular into a single unit configured along the lines of a probe that can be inserted into the tissue. Generation, modification, irradiation and/or detection means configured in such a way can be brought via the cavity essentially directly to the location of the tissue that is to be examined. The local resolution can be further improved. The tissue can be examined with even greater precision.

According to one embodiment of the invention, a light conductor is used to conduct the electromagnetic radiation and/or the luminescent light. A light conductor can be inserted into the cavity via a catheter or a probe. The electromagnetic radiation can thus be directed straight to the tissue. Likewise, the luminescent light generated in the tissue can be directed to the detection means using a light conductor. Absorption and scatter losses in or outside the tissue can be reduced. Even more precise results can be achieved in the examination.

According to a further embodiment of the invention, an image of the tissue is automatically generated with a localized rendition of the intensity of the detected luminescent light or of a value derived therefrom. The localized rendition can be a one-, two-, or three-dimensional rendition. The rendition can be a gray step or false color rendition. The two-dimensional rendition can contain one or a plurality of cross section- or projection images. The localized rendition can be used to establish a diagnosis. The derived value can be a diagnostic parameter selected from the following group: density, electrolyte content, homogeneity, concentration and composition of the electrolytes and of the tissue.

According to a further embodiment of the invention, a computer is used to carry out at least one of steps a) to d) and/or to detect the modifications and/or to generate the rendition. By using a computer, the implementation of the method can be automated and simplified for the user. Furthermore the reliability and precision of the method can be increased, by avoiding user errors, for example.

A further aspect of the invention provides a diagnostic method comprising steps a) to c) and a further step involving the insertion of at least one of the generation, irradiation, modification, and detection means into a cavity located in the tissue or leading to the tissue. The insertion step can be implemented before implementing process steps a) to d). It is also possible to insert the generation, irradiation, modification, and/or detection means before or during the implementation of the respective steps a) to d). Aids known in the prior art, such as catheters, probes etc. can be used for the insertion thereof.

According to a further aspect of the invention, a device is provided for examining a biological tissue, having

a) generation means for the generation of a magnetic field in the tissue such that a permutation symmetry imbalance is generated in a luminescent substance located in the tissue,
b) modification means for the generation of an appropriate continuous or pulsed magnetic alternating field in the tissue such that the permutation symmetry imbalance is modified at a given location,
c) irradiation means for the irradiation of the tissue with continuous or pulsed electromagnetic radiation suitable for stimulating a luminescence of the luminescent substance and
d) a detection means for the detection of a luminescent light generated by the luminescence, depending on the location of the modification in the permutation symmetry imbalance.

The advantages of the method according to the invention and of the embodiments thereof also apply by analogy to the device and to the embodiments of the device.

### BRIEF DESCRIPTION OF THE DRAWINGS

Advantageous embodiments of the invention are described in greater detail below with reference to the figures. The figures show:

FIG. 1 an arrangement for the implementation of the method in a mouse,

FIG. 2 a diagram showing measurement results obtained using the arrangement according to FIG. 1 and

FIG. 3 a diagram showing a further arrangement for the implementation of the method.

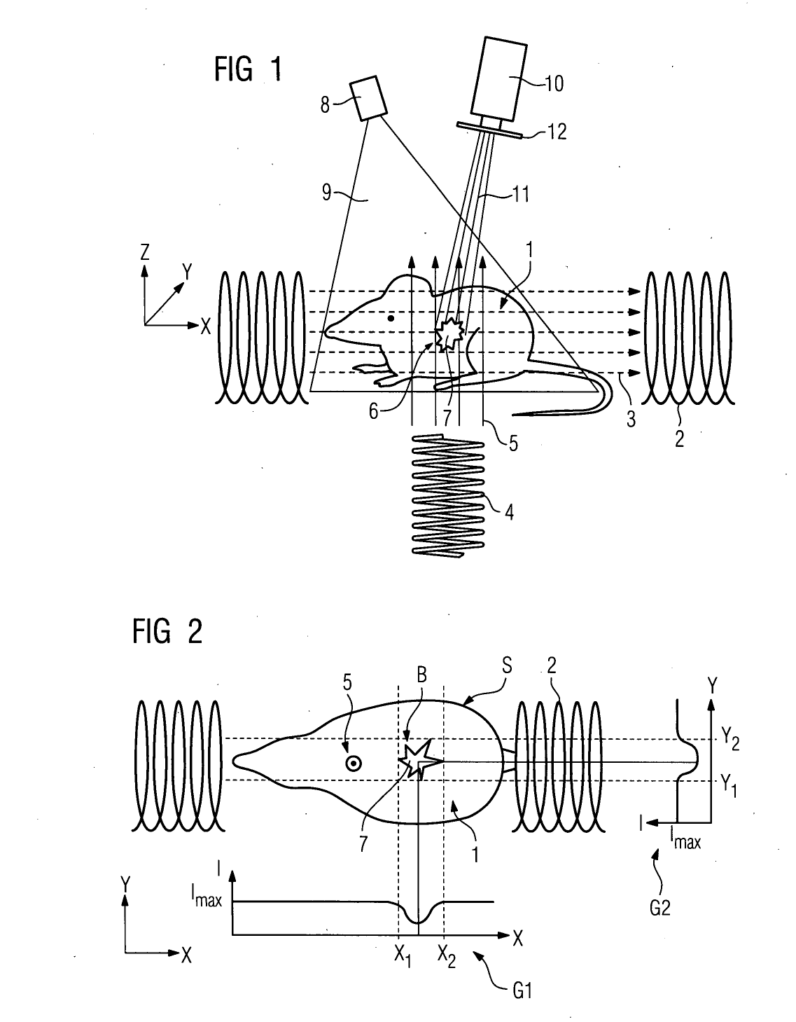

In FIG. 1 to FIG. 3, elements having the same or similar properties are denoted by the same reference signs.

### DETAILED DESCRIPTION OF THE INVENTION

A magnetic field 3 is generated in a tissue 1 of a mouse, using two first magnetic coils 2, for example. A second magnetic coil 4 is used to generate a magnetic alternating field 5 essentially perpendicular to the magnetic field 3. A luminescent substance 7 is accumulated in a tumor 6 located in the tissue 1. An excitation light emanating from a light source 8 to excite the luminescent substance 7 is denoted by the reference sign 9. The reference sign 10 denotes a CCD camera for detecting a luminescent light 11 emanating from the luminescent substance 7. A filter 12 is connected upstream of the CCD camera. X, Y and Z are used to denote an X, Y and Z direction. X1 and X2 and Y1 and Y2 denote first and second X and Y co-ordinates respectively.

The method is implemented as follows:

In a first step, a permutation symmetry imbalance is generated in the tissue 1 of the mouse using the magnetic field 3 generated using the first magnetic coils 2. The permutation symmetry imbalance is generated, for example, by spins aligning themselves in the magnetic field. A permutation symmetry imbalance can involve a permutation symmetry imbalance of nuclear- or electron-spins, for example. The permutation symmetry imbalance can be generated directly or indirectly by linkages, polarization effects or transfer mechanisms.

In a second step, the tissue 1 is irradiated with the excitation light 9. A luminescence of the luminescent substance 7 is excited by the excitation light 9. The luminescent substance 7 can, for example, be a fluorophor which builds up in the tumor 6. It can also be a fluorogen, however, which is activated in the tumor 6 by tumor-specific enzymes, for example, proteases. Furthermore, a fluorochrome or fluorescent molecules which are specifically bound in the tumor 6 can be used. Luminescent substances 7, which can be excited with excitation light 9 in the wavelength region between

200 nm and 2000 nm, preferably between 650 nm and 800 nm, can be used. Such an excitation light 9 is essentially harmless for the tissue 1. Damage or complications caused by the examination can be avoided.

In a third step, a magnetic alternating field 5 suitable for modifying the permutation symmetry imbalance of the luminescent substance 7 is generated at a given location in the tissue 1. As a result of the permutation symmetry imbalance, the probability of the occurrence of radiating and non-radiating transfers in the luminescent substance 7 is modified. The alternating field 5 is generated in the tissue 1 in such a way that the alternating field 5 or at least a component thereof is perpendicular to the magnetic field 3. Furthermore, the frequency of the alternating field 5 fulfils the condition of resonance at a given location.

In a fourth step, the intensity of the luminescent light 11 is detected with the CCD camera 10, depending on the location of the modification in the permutation symmetry imbalance. The intensities detected are recorded and processed by an evaluation means that is not shown, a computer, for example. The given location at which the condition of resonance is fulfilled is used as local information. Furthermore, local information can be acquired from an intensity distribution of the luminescent light 11 generated by the CCD camera 10. In order to further improve the precision of the examination, examination results can be obtained for various arrangements of the first magnetic coils 2 and the second magnetic coils 4, the light source 8 and the CCD camera 10. The intensities detected can finally be used to generate an image of the tissue 1 with a localized resolution of the intensity of the detected luminescent light 11 or of a value derived therefrom. The derived value can be, for example, density, electrolyte content, homogeneity of the tissue 1 or suchlike.

Instead of the intensity of the luminescent light 11, the modification of the intensity of the luminescent light 11 caused by the alternating field 5 can be recorded. In this case, the intensity is detected before and after the generation of the alternating field 5. The modification is recorded with the evaluation means. By recording the modifications in the intensity, errors caused by a reduction in the overall intensity of the luminescent light 11 can be avoided and/or corrected.

It is possible to use the position of the second magnetic coil 4 as additional local information. Local information can also be obtained by using a gradient field as the magnetic field 3. When a gradient field is used, the condition of resonance is fulfilled depending on the location in the tissue 1 and alters in the direction of the gradient. By generating an alternating field 5 with a frequency that corresponds to the condition of resonance at the given location, the permutation symmetry imbalance can be modified locally at the given location. The gradient field itself therefore indirectly contains local information that can be used for localized detection of the luminescent light 11 or for the localized determination of the modifications of luminescence.

The strength of the magnetic field 3 can be constant. To fulfill the condition of resonance, the frequency of the alternating field 5 is modified. It is also possible, however, for the frequency of the alternating field 5 to be constant and for the strength of the magnetic field 3 to be modified.

FIG. 2 shows in diagram form a view of measurement results obtained using the arrangement according to FIG. 1. A section through the mouse running parallel to the X-direction X and Y-direction Y is denoted by the reference sign S. A first graph G1 shows the intensity of the detected luminescent light 11, depending on the location, in the X-direction X. A second graph G2, shows the intensity of the detected luminescent light 11, depending on the location, in the Y-direction Y. A detected maximum intensity is denoted by Iₘₐₓ. The tumor 6 and a fluorogen that can be activated by tumor proteins are contained within a region B located in the section S. First and second X and Y co-ordinates are denoted by the reference signs X1 and X2 and Y1 and Y2 respectively.

The measurement results of the first graph G1 and the second graph G2 are obtained as follows:

Depending on the location in the X-direction X, an alternating field 5 extending over the entirety of the tissue 1 in the Y-direction Y, which alternating field fulfills the condition of resonance, is generated and the intensity of the luminescent light 11 is detected. If the alternating field is generated outside the region B, the fluorescence is not impaired. The maximum intensity Iₘₐₓ of the fluorescent light is detected. If, on the other hand, the alternating field 5 is also generated in the region B, the fluorescence is impaired by a modification of the permutation symmetry imbalance caused by the alternating field 5. As a result thereof, modified intensities are detected between the first X-coordinate X1 and the second X-coordinate X2. The position of the fluorogen and hence of the tumor 6 can be limited in the X-direction X to the interval between the first X-coordinate X1 and the second X-coordinate X2. Likewise, the position of the fluorogen and hence of the tumor 6 can be limited in the Y-direction Y to the interval between the first Y-coordinate Y1 and the second Y-coordinate Y2. An even more precise limitation of the position of the tumor 6 is possible by obtaining further measurement values. A more precise position of the tumor 6 can be obtained, for example, with additional measurement values for the Z-direction Z, for different sections S and different arrangements of the first magnetic coils 2 and the second magnetic coils 4 and of the mouse. It is also possible to alter the arrangement of the light source 8 and the CCD camera 10. For example, the position of the light source 8 and/or of the CCCD camera 10 can be modified by rotating the mouse. Finally it is also possible to use different detectors, light sources 8 of different wavelengths or a plurality of luminescent substances 11.

Measurement results for luminescent substances 7 which are activated by tumor-specific enzymes or specifically bound in the tumor 6 can be obtained in a similar manner. In the case of luminescent substances 7 which accumulate in the tumor 6, a modification of the luminescence can also be caused by the alternating field 5 outside the region B. Said modification differs, as a result, for example, of differences in concentration in the luminescent substance 7, from the modification of the luminescence in the tumor 6. Using the differences, it is possible to locate the tumor 6 in a safe and reliable manner. Instead of the intensity of the luminescent light 11, it is also possible to record modifications in the intensity. Furthermore, it is also possible to generate automatically an image of the tissue 1 with localized resolution of the measurement values, that is, the intensities or a value derived therefrom, the modification of the intensity or a diagnostic parameter.

When implementing the method described in FIG. 1 or FIG. 2, steps a) to c) are implemented in succession. It is possible to change the sequence. The order of steps a) and b) can be changed, for example. A computer can be used to implement the method, preferably in all the steps. The computer can be used to automate the irradiation of the tissue with the excitation light 9, the adjustment of the field intensity of the magnetic field 3, the gradient strength of the gradient field and/or the frequency of the alternating field 5, the detection of the luminescent light 11, the determination of the modifications in the intensity and/or such like. Furthermore, the localized rendition can be generated on a computer. A computer allows a particularly fast and efficient implementation of the method to be achieved. In particular, the implementation of the method can be simplified for a user and errors caused by the user can largely be avoided.

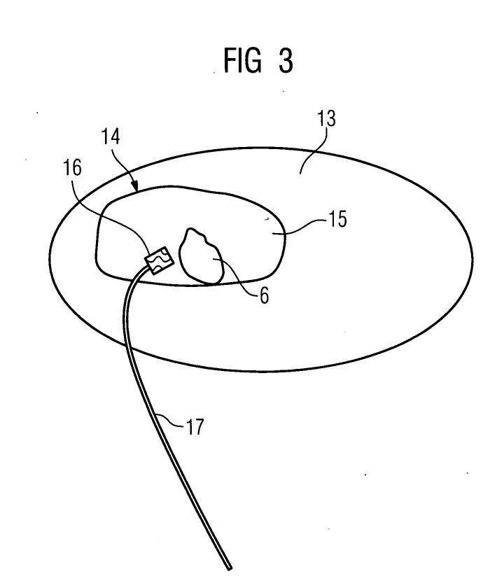

FIG. 3 shows a diagram of a further arrangement for the implementation of the method. An organ 14 located in a patient's body 13 has a tumor 6 that protrudes into a cavity 15 of the organ 14. A measuring unit 16 is inserted into the cavity 15 using a probe 17. For the sake of clarity, the magnetic field 3, the alternating field 5, the luminescent substance 7, the excitation light 9 and the luminescent light 11 are not shown.

The implementation of the examination using the further arrangement proceeds as follows:

The measuring unit 16 comprises generation and modification means to generate or modify a permutation symmetry imbalance in the organ and/or tumor tissue. The measuring unit 16 further has an irradiation means (8) for the irradiation of organ and/or tumor tissue that has been treated with a luminescent substance 7, said means having electromagnetic radiation suitable for stimulating a luminescence of the luminescent substance 7.

Furthermore, the measuring unit 16 comprises a detection means for the detection of a luminescent light (11) emanating from the luminescent substance 7. In order to generate the permutation symmetry imbalance, the generation means can be a coil or a permanent magnet. The modification of the permutation symmetry imbalance can be achieved by the modification means, using coils or electrostatically. The electromagnetic radiation can be generated by irradiation means at the measuring unit 16, for example with a diode. It is also possible for the irradiation means to have a light conductor that runs via the probe 17 to the measuring unit 16. Electromagnetic radiation generated outside the patient's body 13 can be directed to the measuring unit 16 via the light conductor. The detection means can comprise a photodetector or suchlike housed in the measuring unit 16. It is also possible for the detection means to include a light conductor, by means of which the luminescent light 11 emanating from the luminescent substance 7 is directed from the measuring unit 16 to a photodetector or suchlike located outside the body 13. Directing or moving the measuring unit 16 in the cavity 15 can be achieved either manually or automatically by means of incoming lines, outgoing lines or control lines which run from outside the patient's body 13 via the probe 17 to the measuring unit 16. To examine the organ 14, the measuring unit 16 is inserted into the cavity 15 and the method according to steps a) to d) is implemented, the magnetic field 3, the alternating field 6 and the excitation light 9 being generated locally at the measuring unit 16 in the cavity 15. Furthermore, the luminescent light 11 is recorded or detected locally at the measuring unit 16. Any possible influence exerted on the magnetic field 3, the alternating field 6, the excitation light 9 and the luminescent light 11 by layers of tissue surrounding the organ 14 can be reduced. For example, scatter and absorption losses can be considerably reduced, compared to those incurred in the arrangement described in FIG. 1. The organ 14 can be examined with particular precision and the tumor 6 can be located in a particularly safe manner.

It is also possible for the measuring unit 16 to have only one or any combination of the generating, modification, irradiation and detection means. For example, the measuring unit 16 can include the irradiation, the modification and detection means. By analogy with FIG. 1, first magnetic coils 2 located outside the patient's body 13 can be used to generate the magnetic field 3.

A device suitable for the implementation of the method can comprise the components shown in FIG. 1 and FIG. 2. Accordingly, the device can comprise first magnetic coils 2, a second magnetic coil 4, at least one light source 8, and a detector 10 with a filter 12. Furthermore, the device can comprise an evaluation means and/or a computer. A suitable device can also be configured, as shown in FIG. 3, such that the components can be inserted into a cavity 15 located in an organ 14 or generally located in a tissue or cavity 15 leading thereto. With the aforementioned devices, a particularly precise examination of a tissue 1, organ 14 and suchlike is possible. A lesion or, for example, a tumor 6, can be located safely and reliably. The device can be used as an autonomous diagnostic means.

1.-41. (canceled)

42. A method for examining a biological tissue in a mammal, comprising:
generating a magnetic field in the biological tissue for generating a permutation symmetry imbalance in a luminescent substance located in the biological tissue;
irradiating the biological tissue with an electromagnetic radiation for exciting luminescence of the luminescent substance;
generating a magnetic alternating field for modifying the permutation symmetry imbalance in the luminescent substance at a location in the biological tissue; and
detecting a luminescent light generated by the luminescence of the luminescent substance depending on the location of the modification in the permutation symmetry imbalance.

43. The method as claimed in claim 42, wherein a first luminescent light is detected after the step of irradiating and before the step of generating the magnetic alternating field and a modification of the luminescent light caused by the magnetic alternating field is recorded depending on the location of the modification in the permutation symmetry imbalance.

44. The method as claimed in claim 42, wherein the luminescent substance is selected from the group consisting of: fluorophores, fluorochromes, fluorogens, fluorescent molecules, and fluorescent protein.

45. The method as claimed in claim 42,
wherein the electromagnetic radiation is generated by a monochromatic radiation source, and
wherein the monochromatic radiation source is a light having a wavelength of between 200 nm and 2000 nm.

46. The method as claimed in claim 42, wherein the luminescent light is filtered before detecting.

47. The method as claimed in claim 42, wherein the luminescent light is detected by a detector selected from the group consisting of: a CCD camera, an optical sensor, a photodiode, a photoresistor, a phototransistor, a photomultiplier, and a pyroelectric.

48. The method as claimed in claim 42, wherein the magnetic field is a gradient field.

49. The method as claimed in claim 42, wherein the magnetic field and the magnetic alternating field are generated by magnetic coils or permanent magnets.

50. The method as claimed in claim 42, wherein the electromagnetic radiation or the luminescent light is conducted by a light conductor.

51. The method as claimed in claim 42, wherein an image of the biological tissue is automatically generated with a localized rendition of an intensity of the detected luminescent light or of a value derived therefrom.

52. The method as claimed in claim 51, wherein the derived value is a diagnostic parameter selected from the group consisting of: density, electrolyte content, homogeneity, concentration, composition of the electrolytes and of the biological tissue.

53. The method as claimed in claim 42, wherein at least one of the steps is executed by a computer.

54. A device for examining a biological tissue in a mammal, comprising:
a first generator that generates a magnetic field in the biological tissue for generating a permutation symmetry imbalance in a luminescent substance located in the biological tissue;
a second generator that generates a magnetic alternating field in the biological tissue for modifying the permutation symmetry imbalance in the luminescent substance at a given location in the biological tissue;
an irradiator that irradiates the biological tissue by an electromagnetic radiation for stimulating a luminescence of the luminescent substance; and
a detector that detects a luminescent light generated by the luminescence of the luminescent substance depending on the location of the modification in the permutation symmetry imbalance.

55. The device as claimed in claim 54, wherein the first generator, or the second generator, or the irradiator, or the detector is inserted into a cavity located in the biological tissue or a cavity leading to the biological tissue.

56. The device as claimed in claim 54, further comprising a recorder that records a modification of the luminescent light caused by the magnetic alternating field depending on the location of the modification in the permutation symmetry imbalance.

57. The device as claimed in claim 54,
wherein the irradiator comprises a monochromatic radiation source, and
wherein the monochromatic radiation source is a light having a wavelength in a range between 200 nm and 2000 nm.

58. The device as claimed in claim 54, further comprising a filter that is connected upstream of the detector.

59. The device as claimed in claim 54, wherein the detector is selected from the group consisting of: a CCD camera, an optical sensor, a photodiode, a photoresistor, a phototransistor, a photomultiplier, and a pyroelectric detector.

60. The device as claimed in claim 54, wherein the first and the second generators comprise magnetic coils or permanent magnets.

61. The device as claimed in claim 54, wherein the irradiator or the detector comprises a light conductor.
